(19) United States
(12) Patent Application Publication
(10) Pub. No.: US 2008/0226587 A1
(43) Pub. Date: Sep. 18, 2008

(54) METHODS AND COMPOSITIONS FOR IMMUNOMODULATION

(76) Inventors: D. Branch Moody, West Roxbury, MA (US); Ildiko Van Rhijn, Amsterdam (NL); David C. Young, Benton, ME (US); Catherine E. Costello, Reading, MA (US)

Correspondence Address: FISH & RICHARDSON PC, P.O. BOX 1022, MINNEAPOLIS, MN, 55440-1022, US

(21) Appl. No.: 11/866,111

(22) Filed: Oct. 2, 2007

Related U.S. Application Data
(60) Provisional application No. 60/849,224, filed on Oct. 4, 2006.

(51) Int. Cl. A61K 38/19; A61K 38/08; C12N 5/08; G01N 33/68; A61K 38/10; A61P 37/02

(52) U.S. Cl. 424/85.1; 514/17; 514/14; 514/18; 514/16; 514/15; 435/372.3; 435/7.24

(57) ABSTRACT

Compositions for immunomodulation that include acylated peptide antigens recognized by CD1c-restricted T cells, and methods of using the compositions, are provided herein.

### CROSS-REFERENCE TO RELATED APPLICATIONS

This application claims the benefit of priority from U.S. Provisional Patent Application Ser. No. 60/849,224, filed on Oct. 4, 2006, the entire contents of which are incorporated herein by reference.

### FEDERALLY SPONSORED RESEARCH OR DEVELOPMENT

The work described herein was funded, in part, through a grant from the National Institutes of Health (Grant No. AI 49313 awarded to D. Branch Moody). The United States government may, therefore, have certain rights in the invention.

### TECHNICAL FIELD

This invention relates to CD1 antigens, compositions, cells, and methods relating to the use of antigens for immune modulation and diagnosis.

### BACKGROUND

The mammalian immune system is comprised of a complex array of molecular and cellular mediators that recognize and react to microbial antigens. Cell-mediated responses are critical for maintaining immunocompetence. Cell-mediated immune reactions are also thought to be responsible for undesirable responses such as those associated with allergy and certain autoimmune diseases.

T lymphocytes, or T cells, orchestrate cell-mediated immune responses. Antigens derived from polypeptides are presented to T cells through a group of molecules known as major histocompatibility complex (MHC) molecules. MHC molecules are expressed on the surface of cells in association with small peptide ligands. A receptor on T cells (T cell receptor, or TCR) binds to MHC/peptide complexes on the surface of cells. In general, antigens presented by MHC class I molecules are recognized by CD8⁺ T cells, while antigens presented by MHC class II molecules are recognized by CD4⁺ T cells.

Antigen presentation to T cells also occurs through a distinct family of antigen presenting molecules, CD1 molecules. These proteins are displayed on antigen-presenting cells which include Langerhans cells, activated B-cells, dendritic cells in lymph nodes, activated blood monocytes, etc. Although their structures resemble MHC molecules, CD1 molecules differ from MHC molecules in a variety of ways. For instance, CD1 genes are non-polymorphic, while human MHC genes are highly polymorphic. CD1 molecules possess an antigen-binding groove which is deeper than the peptide-binding groove of MHC molecules. The range of antigenic structures presented by CD1 proteins to T cells includes lipids, glycolipids, lipopeptides, and small aromatic compounds. T cells restricted by CD1a, CD1b and CD1c recognize many types of microbial lipids, and are normally activated during the course of acute M. tuberculosis infections in humans. Pathogen recognition can be achieved by TCR recognition of many different antigens produced during infection, so the use of diverse TCRs by CD1-restricted T cells is thought to be relevant for CD1 presented antigens, just like it is for MHC presented antigens.

### SUMMARY

The invention includes a novel class of antigens recognized by CD1-reactive T cells. The novel antigens are acylated peptides presented by CD1 molecules (e.g., CD1c molecules). The antigens are useful, for example, for modulating T cell activity and evaluating immune responses.

Accordingly, in one aspect, the invention features a composition including a compound of formula I: R-GGKWSK (SEQ ID NO: 2), wherein R is an alkyl or alkene chain. The GGKWSK amino acid sequence corresponds to SEQ ID NO:1. In various embodiments, R is an alkyl or alkene chain at least 12, 13, 14, 15, 16, 17, 18, or 20 carbons in length. For example, R is one of the following (expressed as number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. In some embodiments, R is an unsaturated alkyl. In some embodiments, R includes one, two, three, or more double bonds. In certain embodiments, R is C18 or C18:1. In one embodiment, the compound includes the structure of formula II:

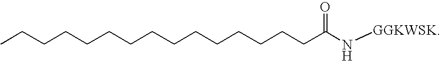

In one embodiment, the composition includes an amount of the compound sufficient to modulate (e.g., increase, or inhibit) an activity of a T cell, such as proliferation. In one embodiment, the composition includes an amount of the compound sufficient to modulate an immune response in a subject. The compound can be a compound that modulates an activity of a CD1 restricted T cell, e.g., a CD1c restricted T cell. In one embodiment, the compound increases an activity of a CD1 restricted T cell, e.g., proliferation, or cytokine release.

In another aspect, the invention features a composition including a compound of formula III: R-GGKWSK-O-KynSKWSK (SEQ ID NO:3); wherein R is an alkyl or alkene chain, and wherein Kyn is kynurenine. In various embodiments, R is an alkyl or alkene chain at least 12, 13, 14, 15, 16, 17, 18, or 20 carbons in length. For example, R is one of the following (expressed as number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. In some embodiments, R is an unsaturated alkyl. In some embodiments, R includes one, two, three, or more double bonds. In certain embodiments, R is C18 or C18:1. In one embodiment, the compound includes the structure of formula IV:

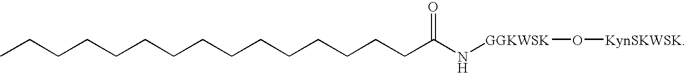

In one embodiment, the composition includes an amount of the compound sufficient to modulate (e.g., increase, or inhibit) an activity of a T cell, such as proliferation. In one embodiment, the composition includes an amount of the compound sufficient to modulate an immune response in a subject. The compound can be a compound that modulates an activity of a CD1 restricted T cell, e.g., a CD1c restricted T cell. In one embodiment, the compound increases an activity of a CD1 restricted T cell, e.g., proliferation, or cytokine release.

In another aspect, the invention features a composition including a glycine-acylated peptide, wherein the peptide is present in an amount sufficient to modulate an activity of a T cell (e.g., T cell proliferation, or cytokine release). For example, the composition is antigenic and/or immunomodulatory.

In one embodiment, the peptide is N-terminally acylated.

In one embodiment, the peptide is present in an amount sufficient to modulate an immune response in a subject.

In one embodiment, the peptide stimulates (e.g., increases) an activity of a T cell (e.g., the peptide stimulates proliferation of a CD1c restricted T cell).

In one embodiment, the peptide inhibits an activity of a T cell (e.g., the peptide inhibits proliferation of a CD1c restricted T cell).

In one embodiment, the peptide is a compound of formula V: R-G-Xₙ; wherein R is an alkyl or alkene, wherein G is glycine, and wherein X is any amino acid. In one embodiment, n=3-15 (e.g., the peptide includes between 3 and 15 amino acid residues, in addition to the N-terminal glycine). For example, n is 4, 5, 6, 7, 8, 9, 10, 11, 12, 13, 14, 15, 16, 17, 18, 19, or 20. In various embodiments, X includes a G, K, W, and/or an S (letters are single letter amino acid abbreviations, and stand for glycine, lysine, tryptophan, and serine, respectively).

In one embodiment, X₄=S or T. In this embodiment, the compound includes the following sequence: G-X₁-X₂-X₃-S (SEQ ID NO: 4) or G-X₁-X₂-X₃-T (SEQ ID NO: 5), wherein X is any amino acid. In one embodiment, X₄ is S or T, and X₁ is G (i.e., the peptide includes the following sequence: G-G-X₂-X₃-S (SEQ ID NO: 6) or G-G-X₂-X₃-T (SEQ ID NO: 7)).

In one embodiment, X₁ is G, A, V, L, I, S, C, T, D, E, or N. For example, X₁ is G.

In various embodiments, n=5 and X₁ is G, A, V, I, L, or M; X₂ is K, R, or H; X₃ is W, F, or Y; X₄ is S or T; and X₅ is K, R, or H.

In various embodiments, the compound includes one of the following sequences: GXKWSK (SEQ ID NO: 8); GGXWSK (SEQ ID NO: 9); GGKXSK (SEQ ID NO: 10); GGKWXK (SEQ ID NO: 11); GGKWSX (SEQ ID NO: 12); wherein X is any amino acid.

In various embodiments, the peptide includes at least 3 amino acid residues, e.g., the peptide includes at least 5 amino acid residues, and, e.g., the peptide has fewer than 50, 25, 15, or 10 amino acid residues. For example, the peptide is 5-10 amino acid residues in length.

In one embodiment, the peptide is a glycine-acylated peptide which is 6 amino acids in length. The acyl modification can include, e.g., an alkyl or alkene chain.

The alkyl or alkene chain of the peptide can be at least 12, at least 13, at least 14, at least 15, at least 16, at least 17 or at least 18 carbons in length. For example, the alkyl or alkene chain is one of the following (number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. The alkyl or alkene can be an unsaturated alkyl, or can include at least one, two, or three double bonds. In some embodiments, the alkyl chain is a saturated alkyl chain with a chain of at least 16 carbons.

In one embodiment, the peptide is a microbial peptide or fragment thereof. For example, the peptide is a viral peptide or fragment thereof (e.g., an HIV peptide or fragment thereof, such as a Nef peptide or a Gag peptide; a peptide of a herpes virus, such as HSV-1), a fungal peptide or fragment thereof, a bacterial peptide or fragment thereof, or a parasitic peptide or fragment thereof.

In still other embodiments, the peptide is a mammalian peptide or fragment thereof (e.g., an autoantigen, or a tumor antigen). In some embodiments, the peptide includes an allergen.

In some embodiments, the peptide is a peptide which, when naturally expressed in a cell, is acylated. In other embodiments, the peptide is not naturally expressed in an acylated form, and can be produced by synthetic means.

In one embodiment, the peptide is a Nex antigen described herein.

The composition including a glycine-acylated peptide can further include a second compound. In one embodiment, the second compound includes a second peptide or non-peptide antigen. In one example, the glycine-acylated peptide and the second peptide or non-peptide antigen are from the same microbe (e.g., the glycine-acylated peptide is an HIV peptide and the second peptide is also an HIV peptide; or the glycine-acylated peptide is a bacterial peptide and the second peptide or non-peptide antigen is from the same bacterial species).

In one embodiment, the second compound includes an immunomodulatory agent. The immunomodulatory agent includes, for example, a cytokine (e.g., an interleukin such as IL-4 or IL-2; GM-CSF; or G-CSF), an adjuvant (e.g., alum, Freund's adjuvant, mycobacterial cell wall components), or an immunosuppressive drug (e.g., cyclosporine A, or a steroid).

The invention features an immunogenic compositions (e.g., a vaccines) including a glycine-acylated peptide as described herein, wherein the peptide is present in an amount sufficient to modulate proliferation of a T cell.

In another aspect, the invention features a method for modulating activity of a T cell. The method includes: contacting the T cell with a composition including an antigen-presenting cell (APC) and a glycine-acylated peptide, thereby modulating the activity of the T cell. For example, the APC expresses CD1 molecules (e.g., CD1c molecules).

In one embodiment, the peptide is N-terminally acylated. In one embodiment, the peptide is a compound of formula V: R-G-Xₙ; wherein R is an alkyl or alkene, wherein G is glycine, wherein X is any amino acid, and wherein n is 3-15. For example, n is 4, 5, 6, 7, 8, 9, 10, 11, 12, 13, 14, 15, 16, 17, 18, 19, or 20.

In various embodiments, the peptide includes at least 3 amino acid residues, e.g., the peptide includes at least 5 amino acid residues, and, e.g., the peptide has fewer than 50, 25, 15, or 10 amino acid residues. For example, the peptide is 5-10 amino acid residues in length.

In one embodiment, the peptide is a glycine-acylated peptide which is 6 amino acids in length. The acyl modification can include, e.g., an alkyl or alkene chain.

The alkyl or alkene chain of the peptide can be at least 12, at least 13, at least 14, at least 15, at least 16, at least 17 or at least 18 carbons in length. For example, the alkyl or alkene chain is one of the following (number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. The alkyl or alkene can be an unsaturated alkyl, or can include at least one, two, or three double bonds. In some embodiments, the alkyl chain is a saturated alkyl chain with a chain of at least 16 carbons.

In one embodiment, the peptide is a microbial peptide or fragment thereof. For example, the peptide is a viral peptide or fragment thereof (e.g., an HIV peptide or fragment thereof, such as a Nef peptide or a Gag peptide), a fungal peptide or fragment thereof, or a bacterial peptide or fragment thereof, or a parasitic peptide or fragment thereof.

In still other embodiments, the peptide is a mammalian peptide or fragment thereof (e.g., an autoantigen, or a tumor antigen). In some embodiments, the peptide includes an allergen.

In some embodiments, the peptide is a peptide which, when naturally expressed in a cell, is acylated. In other embodiments, the peptide is not naturally expressed in an acylated form, and can be produced by synthetic means.

The method can further include a use of a second compound. In one embodiment, the second compound includes a second peptide or non-peptide antigen. In one example, the glycine-acylated peptide and the second peptide or non-peptide antigen are from the same microbe (e.g., the glycine-acylated peptide is an HIV peptide and the second peptide is also an HIV peptide; or the glycine-acylated peptide is a bacterial peptide and the second peptide or non-peptide antigen is from the same bacterial species).

In one embodiment, the second compound includes an immunomodulatory agent. The immunomodulatory agent includes, for example, a cytokine (e.g., an interleukin such as IL-4 or IL-2; GM-CSF; or G-CSF), an adjuvant (e.g., alum, Freund's adjuvant, mycobacterial cell wall components), or an immunosuppressive drug (e.g., cyclosporine A, or a steroid).

In various embodiments, the acylated peptide is a compound of formula I, II, III, or IV.

In various embodiments of the method, T cell activity is modulated in a subject in vivo, ex vivo, or in vitro. In various embodiments, the subject is at risk for, being screened for, or is diagnosed with an infection, an autoimmune disorder, an allergic disorder, or a neoplastic disorder. The subject may be immunocompromised.

The invention also features a method for modulating an immune response in a subject. The method includes identifying a subject in need of modulation of an immune response, and administering to the subject a composition including a glycine-acylated peptide in an amount effective to modulate an immune response.

In one embodiment, the peptide is N-terminally acylated. In one embodiment, the peptide is a compound of formula V: R-G-Xₙ; wherein R is an alkyl or alkene, wherein G is glycine, wherein X is any amino acid, and wherein n is 3-15. For example, n is 4, 5, 6, 7, 8, 9, 10, 11, 12, 13, 14, 15, 16, 17, 18, 19, or 20.

In various embodiments, the peptide includes at least 3 amino acid residues, e.g., the peptide includes at least 5 amino acid residues, and, e.g., the peptide has fewer than 50, 25, 15, or 10 amino acid residues. For example, the peptide is 5-10 amino acid residues in length.

In one embodiment, the peptide is a glycine-acylated peptide which is 6 amino acids in length. The acyl modification can include, e.g., an alkyl or alkene chain.

The alkyl or alkene chain of the peptide can be at least 12, at least 13, at least 14, at least 15, at least 16, at least 17 or at least 18 carbons in length. For example, the alkyl or alkene chain is one of the following (number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. The alkyl or alkene can be an unsaturated alkyl, or can include at least one, two, or three double bonds. In some embodiments, the alkyl chain is a saturated alkyl chain with a chain of at least 16 carbons.

In one embodiment, the peptide is a microbial peptide or fragment thereof. For example, the peptide is a viral peptide or fragment thereof (e.g., an HIV peptide or fragment thereof, such as a Nef peptide or a Gag peptide), a fungal peptide or fragment thereof, or a bacterial peptide or fragment thereof, or a parasitic peptide or fragment thereof.

In still other embodiments, the peptide is a mammalian peptide or fragment thereof (e.g., an autoantigen, or a tumor antigen). In some embodiments, the peptide includes an allergen.

In some embodiments, the peptide is a peptide which, when naturally expressed in a cell, is acylated. In other embodiments, the peptide is not naturally expressed in an acylated form, and can be produced by synthetic means.

The method can further include a use of a second compound. In one embodiment, the second compound includes a second peptide or non-peptide antigen. In one example, the glycine-acylated peptide and the second peptide or non-peptide antigen are from the same microbe (e.g., the glycine-acylated peptide is an HIV peptide and the second peptide is also an HIV peptide; or the glycine-acylated peptide is a bacterial peptide and the second peptide or non-peptide antigen is from the same bacterial species).

In one embodiment, the second compound includes an immunomodulatory agent. The immunomodulatory agent includes, for example, a cytokine (e.g., an interleukin such as IL-4 or IL-2; GM-CSF; or G-CSF), an adjuvant (e.g., alum, Freund's adjuvant, mycobacterial cell wall components), or an immunosuppressive drug (e.g., cyclosporine A, or a steroid).

In various embodiments, the acylated peptide is a compound of formula I, II, III, or IV.

In another aspect, the invention features an isolated CD1-reactive T cell (e.g., a human T cell), wherein the CD1-reactive T cell is specific for an acylated peptide. In one embodiment, the T cell is CD1c-reactive. T cell can include an αβ T cell receptor.

In one embodiment, the T cell expresses a T cell receptor encoded by a human T cell receptor beta V12-3 gene segment. In one embodiment, the T cell expresses a T cell receptor encoded by a human T cell receptor alpha V25 gene segment.

In another aspect, the invention features a method for identifying a T cell antigen. The method includes, for example: providing a sample including an antigen-presenting cell (APC)(e.g., a human APC), wherein the APC expresses CD1 molecules (e.g., CD1c molecules); contacting the sample with a composition comprising a glycine-acylated peptide under conditions in which acylated peptides bind to CD1 molecules; contacting the sample with a CD1-restricted T cell (e.g., a CD1c-restricted T cell; and determining activity of the T cell in the presence of the sample, wherein a change in activity of the T cell in the presence of the sample, relative to a control, indicates that the peptide is a T cell antigen.

In one embodiment, the glycine-acylated peptide is N-terminally acylated. In one embodiment, the peptide is a compound of formula V: R-G-Xₙ; wherein R is an alkyl or alkene, wherein G is glycine, wherein X is any amino acid, and wherein n is 3-15. For example, n is 4, 5, 6, 7, 8, 9, 10, 11, 12, 13, 14, 15, 16, 17, 18, 19, or 20.

In various embodiments, the peptide includes at least 3 amino acid residues, e.g., the peptide includes at least 5 amino acid residues, and, e.g., the peptide has fewer than 50, 25, 15, or 10 amino acid residues. For example, the peptide is 5-10 amino acid residues in length.

In one embodiment, the peptide is a glycine-acylated peptide which is 6 amino acids in length. The acyl modification can include, e.g., an alkyl or alkene chain.

The alkyl or alkene chain of the peptide can be at least 12, at least 13, at least 14, at least 15, at least 16, at least 17 or at least 18 carbons in length. For example, the alkyl or alkene chain is one of the following (number of carbons: number of unsaturated bonds): C20, C20:1, C19, C19:1, C18, C18:1, C17, C17:1, C16, C16:1, C15, C15:1, C14, C14:1, C13, C13:1, C12 and C12:1. The alkyl or alkene can be an unsaturated alkyl, or can include at least one, two, or three double bonds. In some embodiments, the alkyl chain is a saturated alkyl chain with a chain of at least 16 carbons.

In one embodiment of the method, the peptide is a microbial peptide or fragment thereof. For example, the peptide is a viral peptide or fragment thereof (e.g., an HIV peptide or fragment thereof, such as a Nef peptide or a Gag peptide), a fungal peptide or fragment thereof, or a bacterial peptide or fragment thereof, or a parasitic peptide or fragment thereof.

In still other embodiments of the method, the peptide is a mammalian peptide or fragment thereof (e.g., an autoantigen, or a tumor antigen). In some embodiments, the peptide includes an allergen.

In some embodiments, the peptide is a peptide which, when naturally expressed in a cell, is acylated. In other embodiments, the peptide is not naturally expressed in an acylated form, and can be produced by synthetic means.

The method can further include purifying the peptide from the composition (e.g., by fractionation and further testing for T cell-modulatory activity).

The invention also features a method for evaluating an immune response in a subject. The method includes, for example, providing a sample from the subject (e.g., a sample of peripheral blood leukocytes), wherein the sample includes T cells; contacting the sample with a composition including an antigen-presenting cell (APC) and a glycine-acylated peptide; evaluating activity of the T cells, relative to a control, thereby evaluating an immune response in the subject.

The glycine acylated peptide can include features described herein. For example, the peptide is N-terminally acylated. In one embodiment, the peptide is a compound of formula V: R-G-Xₙ; wherein R is an alkyl or alkene, wherein G is glycine, wherein X is any amino acid, and wherein n is 3-15. For example, n is 4, 5, 6, 7, 8, 9, 10, 11, 12, 13, 14,15,16,17, 18, 19, or 20.

In various embodiments of the method, the peptide includes at least 3 amino acid residues, e.g., the peptide includes at least 5 amino acid residues, and, e.g., the peptide has fewer than 50, 25, 15, or 10 amino acid residues. For example, the peptide is 5-10 amino acid residues in length.

In one embodiment, the peptide is a glycine-acylated peptide which is 6 amino acids in length. The acyl modification can include, e.g., an alkyl or alkene chain.

The alkyl or alkene chain of the peptide can be at least 12, at least 13, at least 14, at least 15, at least 16, at least 17 or at least 18 carbons in length. For example, the alkyl or alkene chain is one of the following (number of carbons: number of unsaturated bonds): C20, C20:1, C19, C19:1, C18, C18:1, C17, C17:1, C16, C16:1, C15, C15:1, C14, C14: 1, C13, C13: 1, C12 and C12: 1. The alkyl or alkene can be an unsaturated alkyl, or can include at least one, two, or three double bonds. In some embodiments, the alkyl chain is a saturated alkyl chain with a chain of at least 16 carbons.

In one embodiment, the peptide is a microbial peptide or fragment thereof. For example, the peptide is a viral peptide or fragment thereof (e.g., an HIV peptide or fragment thereof, such as a Nef peptide or a Gag peptide), a fungal peptide or fragment thereof, or a bacterial peptide or fragment thereof, or a parasitic peptide or fragment thereof.

In still other embodiments, the peptide is a mammalian peptide or fragment thereof (e.g., an autoantigen, or a tumor antigen). In some embodiments, the peptide includes an allergen.

In some embodiments, the peptide is a peptide which, when naturally expressed in a cell, is acylated. In other embodiments, the peptide is not naturally expressed in an acylated form, and can be produced by synthetic means.

In one embodiment of the method, the immune response of the subject to a microbe (e.g., a virus, bacteria, fungus, or parasite) is evaluated.

In one embodiment of the method, the immune response of the subject to an allergen is evaluated.

In one embodiment of the method, the immune response of the subject to a self antigen is evaluated (e.g., a tumor antigen, or an autoantigen implicated in an autoimmune disorder).

The invention also features compositions for evaluating an immune response of a subject. The compositions include, for example, a glycine-acylated peptide and instructions for using the peptide in an assay to detect immune reactivity (e.g., a T cell-mediated reactivity) of a cell of the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a graph depicting the proliferative response of polyclonal Next T cells stimulated by dendritic cells (DC) and antigen. IL-2 sensitive HT-2 cells were used to measure the response of the polyclonal Next T cells. The proliferative response of cells is depicted as levels of [³H]-thymidine incorporation (antigen concentration, x-axis; counts per minute (cpm), y-axis).

FIG. 1B is a bar graph depicting the proliferative response of Next T cells stimulated in the presence of DC, antigen, and monoclonal antibodies that block CD1a, CD1b, CD1d, control antibody (P3, isotype matched control), or no antibody.

FIG. 1C is a graph depicting the proliferative response of Next T cells stimulated in the presence of antigen and CD1-deficient C1R B lymphoblastoid cells transfected with CD1a, CD1b, CD1c, CD1d, or mock transfected C1R cells.

FIG. 2A is graph depicting the relative abundance of Nex1, Nex2, and a third compound purified from a mixture of synthetic lipopeptide antigens using high performance liquid chromatography with simultaneous electrospray ionization mass spectrometry. Total ion current is the basis for the relative abundance plotted on the y-axis.

FIG. 2B is a graph depicting the proliferative response of Next T cells stimulated with DC and each of the three purified compounds, or no antigen.

FIG. 3A is a graph depicting collisional MS analysis of Nex2.

FIG. 3B is a graph depicting the proposed structure of Nex2 based on collisional MS analysis.

FIG. 4A is a graph depicting the proliferative response of Next T cells stimulated with an untreated Nex antigen mixture, Nex treated with proteinase K, Nex treated with pronase, mock treated Nex, or no antigen.

FIG. 4B is a graph depicting the proliferative response of DDM-reactive J.RT3/CD8-2 cells stimulated in the presence of mock treated DDM, DDM treated with proteinase K, DDM treated with pronase, or no antigen.

FIG. 4C is a bar graph depicting the proliferative response of Next T cells stimulated by mixtures of untreated Nex antigen and Nex antigen treated with proteinase K or pronase.

FIG. 4D is a graph depicting the proliferative response of Next T cells stimulated with CD1c-transfected C1R cells and Nex antigen in the presence and absence of LHVS, a protease inhibitor.

Like reference symbols in the various drawings indicate like elements.

### DETAILED DESCRIPTION

The invention is based, in part, on the discovery of a novel class of antigens recognized by CD1-reactive T cells. The antigens include acylated peptides, such as peptides including an N-terminal acyl modification, e.g., N-terminally glycine acylated peptides. These peptides are presented to T cells on CD1 molecules (e.g., CD1c molecules) and are useful in modulating and evaluating immune responses in which CD1-reactive T cells are implicated. One advantage of the novel antigens described herein lies in the fact that these antigens are presented on CD1 molecules, which are non-polymorphic. Thus, responses to the antigens may be less variable among different subjects (for example, as compared to responses to antigens presented on highly polymorphic MHC molecules).

As used herein an “antigen” is a molecule or composition of matter which induces an immune response in an animal. A “foreign antigen” is one that is not endogenously derived in a normal, healthy animal. In an unhealthy animal, however, endogenous molecules or compositions of matter that are expressed as a result of a condition or disease (e.g. cancer, etc.) can be recognized by the immune system as being foreign. Antigens of the invention also include “autoimmune antigens” which are normal, endogenously derived molecules or compositions of matter in an otherwise normal, is healthy animal. Autoimmune antigens are also commonly referred to as “self antigens” or “autoantigens”.

### CD1 Molecules

CD1 molecules are known to present non-peptide antigens such as lipids and glycolipids. These molecules encoded by the genes of the CD1 locus are recognized by selecting CD4⁻CD8⁻T cell clones expressing either α:β or γ:δ TCRs (Porcelli, S., et al., Nature 341:447-450,1989; Faure, F., et al., Eur. J. Immun. 20:703-706,1990). Because of the structural resemblance of CD1 molecules, encoded by genes on human chromosome 1, to MHC molecules, encoded by genes on human chromosome 6 (Calabi, F. and Milstein, C., Nature 323:540-543,1986; Balk, S. P., et al., Proc. Natl. Acad. Sci. USA 86:252-256,1989), it has been suggested that CD1 may represent a family of antigen presenting molecules separate from those encoded by the MHC genes (Porcelli, S., et al., Nature 341:447-450,1989; Strominger, J. L., Cell 57:895-898,1989; Porcelli, S., et al., Immun. Rev. 120:137-183,1991).

The five CD1 genes reveal exon and domain structure ((α1, α2, α3) that is similar to that of MHC class I genes, yet the proteins are only distantly related in sequence. All CD1 family members share a conserved α3 domain; however, even this domain shows only 32% homology in amino acid sequence with consensus residues of class I MHC α3 domains. A major difference between MHC and CD1 molecules is polymorphism. Human MHC genes are extremely polymorphic: multiple alleles have been described at each known MHC locus. In contrast, CD1 genes are apparently non-polymorphic. Despite these differences, the CD1 molecules, like MHC Class I molecules, are expressed as large subunits (heavy chains) non-covalently associated with β₂-microglobulin (Van Agthoven, A., and Terhorst, C., J. Immunol. 128:426-432,1982; Terhorst, C., et al., Cell 23:771-780,1981).

Four of the five CD1 gene products expressed in humans have been defined serologically, are referred to as CD1a, CD1b, CD1c and CD1d, and are distinguished by unique heavy chains with approximate molecular weights of 49 kDa, 45 kDa, 43 kDa and 48 kDa respectively (Amiot, M., et al., J. Immunol. 136:1752-1758,1986; Porcelli, S., et al., Immunol. Rev. 120:137-183, 1991; Bleicher, P. A., et al., Science 250:679-682,1990). CD1 molecules are displayed on a number of antigen presenting cells (APCs) including Langerhans cells (which are the major dendritic antigen-presenting cells in the skin), activated B-cells, dendritic cells in lymph nodes, and on activated blood monocytes (Porcelli, S., et al., Nature 360:593-597,1992; Leukocyte Typing IV, Knapp, W., ed., Oxford University Press, Oxford, U.K., pp. 251-269, 1989; Tissue Antigens, Kissmeyer-Nielsen, F., ed., Munksgard, Copenhagen, Denmark, pp. 65-72, 1989).

Presentation of the antigens described herein is associated with a CD1c molecule and, thus, are referred to as being “CD1c-restricted”. As used herein, “CD1-restricted antigen” refers to an antigen which is bound by and/or presented with a member of the CD1 family and displayed on the surface of an antigen presenting cell (APC) that expresses the CD1 molecule (also referred to as CD1⁺ cell). The term “CD1-presented antigen” can also be used in place of “CD1-restricted antigen”. In addition, when the antigen is bound to a CD1 molecule the antigen can be referred to as “CD1-bound antigen”.

As used herein, “displayed” refers to the process of localizing a protein, such as a CD1, or a protein:antigen complex, to the outermost surface of a cell where the protein or protein:antigen complex is accessible to a second cell or to molecules displayed by a second cell. In some instances, antigens are processed with cellular factors in order to be made competent for displaying by an APC. “Antigen presenting cell” is a term well known in the art to include cells which present antigen to T cells by way of MHC class I molecules, MHC class II molecules, and/or CD1 molecules. One skilled in the art can use procedures known in the art for determining whether a cell is expressing one or more members of the CD1 family of proteins (see U.S. Pat. Nos. 5,679,347; 5,853,737 and 6,238,676 and Porcelli, S., Immun. Rev. 120:137-183, 1991).

The invention, in part, provides several CD1 antigens identified and isolated from synthetic mixtures. The term “isolated” as used herein refers to a molecular species which is substantially free of proteins, lipids, carbohydrates or other materials with which it is normally associated. One skilled in the art can purify acylated peptides, using standard techniques purification such as those described herein. In various embodiments, a purified composition includes at least 90%, 95%, 96%, 97%, 98%, or 99% of the acylated peptide of interest.

### Novel CD1 Antigens

New antigens have been found to be presented by CD1 molecules, in particular CD1c molecules. These antigens represent a new class of T cell antigens. The antigens are acylated peptides such as glycine-acylated peptides, in which the acylation is on the N-terminal residue.

The acylated peptides include amino acid sequences of various lengths. Exemplarypeptides include 4, 5, 6, 7, 8, 9, 10, 11, 12, 13, 14, 15, 16, 17, 18, 19, or 20 amino acid residues. The acyl modification can include, e.g., an alkyl or alkene chain, such as an alkyl or alkene chain at least 12, at least 13, at least 14, at least 15, at least 16, at least 17 or at least 18 carbons in length. For example, the alkyl or alkene chain is one of the following (number of carbons: number of unsaturated bonds): C20, C20: 1, C19, C19:1, C18, C18:1, C17, C17:1, C16, C16:1, C15, C15:1, C14, C14:1, C13, C13:1, C12 and C12:1. The alkyl or alkene can be an unsaturated alkyl, or can include at least one, two, or three double bonds. In some embodiments, the alkyl chain is a saturated alkyl chain with a chain of at least 16 carbons. Acyl modifications with branched and unbranched carbon chains are contemplated.

The peptide sequences may be derived from microbial peptides (e.g., viral, bacterial, fungal, or parasitic peptides). Many microbes encode peptides which are acylated. For example, the amino terminal fragments of Gag proteins of retroviruses such as HIV are myristoylated (Mervis et al., J. Virol. 62(11):3993-4002, 1988). The Nef protein of HIV is also myristoylated (Peng et al., Immunol Lett. 78(3):195-200, 2001). Accordingly, acylated Nef and Gag polypeptides, acylated fragments of these polypeptides, and derivatives thereof, are contemplated.

The peptide sequences may also be mammalian (e.g., human) peptides. For example, tumor antigens, or autoantigens associated with autoimmune responses are provided. Peptide sequences which are allergens are also contemplated.

In some embodiments, the peptide is a peptide which, when naturally expressed in a cell, is acylated. Co-translationally glycine-acylated peptides typically include a methionine-glycine sequence at the N-terminus. N-myristoylation is catalyzed by N-myristoyl transferase in vivo. Eukaryotic proteins known to be N-myristoylated include G protein α subunits, enzymes, immunoglobulins, and growth factors, among others (Sankaram, Biophys. J. 67:105-112, 1994). Frequently, N-terminal glycine acylated peptides include a serine or threonine as the fifth residue (i.e., the peptides include a G-X-X-X-S/T sequence)(Sankaram, Biophys. J. 67:105-112, 1994). Acylated sequences are described herein and are known in the art. Exemplary acylated peptides include the following amino acid sequences: GGKWSK (SEQ ID NO: 1), GGDASGE (SEQ ID NO: 13), GRGDTP (SEQ ID NO: 14), and variants thereof.

In some embodiments, the peptide is not naturally expressed in an acylated form and is acylated in vitro. The peptides described herein can be produced by synthetic means.

Synthetic antigens may include those derived from nature that have been subsequently manipulated or modified. Alternatively, they include antigens that have no naturally occurring counterparts. For example, the synthetic antigen may contain amino acids, peptide linkages, or lipid linkages not typically found in a cell.

Methods for isolating the new antigens from a sample are known in the art. As used herein, a “sample” is any solution, emulsion, suspension, or extract which can be tested. Like above, a sample can be first fractionated (subjected to conditions or procedures which separate the components of the sample based on physical or chemical properties such as, but not limited to, size, charge, solubility, or composition) using conventional procedures. Examples of procedures include, but are not limited to, selective precipitation, organic extraction, normal or reverse phase high performance chromatography, and ion exchange chromatography. The fractions of the sample are then tested for the presence of the antigen. The antigens may also be isolated by relying on the binding of CD1c with an antigen of the invention. The CD1c may be purified (and cell free) or it may be cell bound. In one example, a sample containing the antigen is contacted with a purified CD1c. If the CD1c is cell bound, and if the cell is activatable upon binding of CD1c by an antigen, then the antigen can be isolated based on its ability to bind to and activate a CD1c-restricted T cell line (e.g., by exposing a CD1c-expressing antigen presenting cell to an antigen of the invention). As used herein, “contacting” is the process of combining one or more entities. The resulting antigen:CD1c complex or antigen:CD1c⁺ cell complex is then separated and further analyzed (e.g., by dissociating the antigen from the CD1c). Alternatively, the complexes can be further screened for their ability to activate CD1c-restricted T cells. Using such a procedure, a purified CD1c-presented antigen is obtained. To further purify the antigens, either type of complex is treated under appropriate conditions such that the CD1c-bound antigen will be released from the CD1c molecule. The CD1c-presented antigens of the present invention can be purified over a wide range of purities. A skilled artisan will know to employ various purification strategies in order to obtain an antigen which has been purified to the extent required for an intended use.

In addition to the above isolation methods, the antigens provided can be synthesized de novo, as described below in the Examples.

The antigens provided herein were identified with T cell activation assays performed using purified fractions in order to determine their relative ability to activate T cells. These assays were carried out essentially as described in U.S. Pat. Nos. 5,679,347; 5,853,737; and 6,238,676, and in Rosat et al. J. Immunol. 162:366-371, 1999. Other procedures are well-known in the art.

A “CD1-restricted T cell” is a mature peripheral blood lymphocyte that expresses T cell antigen receptors, or is TCR⁺. CD1-restricted T cells can recognize a CD1-presented antigen. CD1-restricted T cells can be CD4⁻T cells which are mature peripheral blood TCR⁺ lymphocytes which do not express CD4. Techniques for identifying CD4⁻ T cells are well known in the art and can readily be employed in the present invention, for example in U.S. Pat. Nos. 5,679,347; 5,853,737 and 6,238,676 and in Panchomoorthy, G., et al., J. Immunol. 147:3360-3369,1991).

Other methods of characterizing classes of T cells, and of isolating subpopulations of T cells, have been described. Wysocki, L. J., and Sato, V. L., Proc Natl Acad Sci. USA 75:2844-2848,1978; Wasik, M. A., and Morimoto, C., J Immunol. 144:3334-3340, 1990; Harriman, G. R., et al., J Immunol. 145:4206-2414, 1990; Koulova, L., et al., J Immunol. 145:2035-2043,1990. Methods of culturing T cells in vitro, and of immortalizing T cells via fusion to non-growth restricted cells such as myelomas, have been described. Paul, W. E., et al., Nature 294:697-699, 1981; Williams, N., Nature 296:605-606,1982. T cell populations can be enriched to obtain isolated T cell clones which are reactive to CD1c-presented antigens. A population of T cells is allowed to divide and a subpopulation of mixed T cells is isolated based on proliferation in the presence of CD1⁺ APCs and CD1-presented antigen, or on cytolytic activity against transfected cells expressing CD1 molecules in the presence of a CD1-presented antigens.

### Methods of Use

The novel acylated peptide compounds and antigenic compositions described herein can be used in a number of ways. For example, they are useful as antigens, adjuvants and immunomodulators. Generally, the acylated peptides act at least in part by modulating (e.g., inducing, or inhibiting) a CD1 immune response, and in particular a CD1c immune response. A “CD1 immune response”, as used herein, is an immune response that involves antigen presentation by an antigen presenting cell that expresses a CD1 molecule on its surface to a T cell that recognizes the presented antigen via its TCR. T cells that recognize antigen presented in the context of CD1 become activated as a result, and may respond in a number of ways. For example, these T cells can lyse target cells (e.g., cells infected with bacteria, such as mycobacteria, cells infected with viruses, or cells presenting antigens related to Nex). T cells also respond by secreting γ-interferon which in turn can polarize an immune response towards a Th1 response. The antigens therefore are useful in generating effector T cells.

Previous work has shown also that CD1 molecules are recognized by CD4⁻CD8⁻T cell lines derived from patients with SLE (Porcelli, et al., Nature 341:447-450,1989). Leukemia cells expressing CD1 molecules were lysed by the T cells independent of MHC restriction, even though no foreign (non-self) antigen was present. The T cells lysed leukemic cells in a CD1-dependent manner in the absence of antigen. Thus, CD1 molecules may play a role in autoimmune diseases.

The CD1 antigens can also be used to modulate an immune response. To “modulate an immune response” as used herein means to enhance or inhibit a pre-existing immune response, to stimulate a non-existent immune response, and/or to alter the characteristics of an immune response. Inhibiting an immune response means that the immune response is lessened from a pre-treatment level, and may include but is not limited to a complete abrogation of an immune response. When the lipopeptide antigens are used to enhance the immunity of a subject, it is intended that the antigens can enhance a pre-existing immune response and/or stimulate a non-existent immune response. If a subject has an infection such as a viral or bacterial infection, then the antigen provided herein may be used to stimulate an immune response and/or enhance a pre-existing immune response. If a subject is undergoing an inappropriate immune response that is associated with the antigen, then administration of the antigen may be used to inhibit or alter the characteristics of the immune response. Altering the characteristics of an immune response can include switching an immune response from a Th2 immune response to a Th1 immune response, or vice versa. As used herein, the term “inhibit” means a reduction in symptoms associated with a condition, or complete elimination of the condition, as determined by a medical practitioner.

In some embodiments, the acylated peptide antigens described herein are useful as adjuvants. In other embodiments, acylated peptide compounds are administered together with another agent that is an adjuvant. In these aspects, an adjuvant is any molecule or compound which can stimulate the humoral and/or cellular immune response. Adjuvants include, for instance, adjuvants that create a depo effect, immune stimulating adjuvants, adjuvants that create a depo effect and stimulate the immune system, and mucosal adjuvants. In some embodiments, the adjuvants is preferably an immune stimulating adjuvant.

An “adjuvant that creates a depo effect” as used herein is an adjuvant that causes an antigen to be slowly released in the body, thus prolonging the exposure of immune cells to the antigen. Examples include but are not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

An “immune stimulating adjuvant” is an adjuvant that causes activation of a cell of the immune system. It may, for instance, cause an immune cell to produce and secrete cytokines. Examples include but are not limited to saponins purified from the bark of the Q. saponaria tree, such as QS21 (a glycolipid that elutes in the 21^{st} peak with HPLC fractionation; Antigenics Inc. Woburn, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; O M Pharma S A, Meyrin, Switzerland); and Leishmania elongation factor (a purified Leishmania protein; Corixa Corporation, Seattle, Wash.).

An “adjuvant that creates a depo effect and stimulates the immune system” is a compound that has both of the above-identified functions. Examples include but are not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “mucosal adjuvant” as used herein is an adjuvant that is capable of inducing a mucosal immune response in a subject when administered to a mucosal surface in conjunction with an antigen. Examples include but are not limited to bacterial toxins: e.g., Cholera toxin (CT), CT derivatives including but not limited to CT B subunit (CTB) (Wu et al., Vaccine. 16(2-3):286-92, 1998; Tochikubo et al., Vaccine. 16(2-3):150-5 1998); CTD53 (Val to Asp) (Fontana et al., Infect Immun. 63(6):2356-60, 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., J Exp Med., 185(7):1203-10, 1997); CTS61F (Ser to Phe) (Yamamoto et al., Proc Natl Acad Sci U S A. 94(10):5267-72 ,1997); CTS106 (Pro to Lys) (Douce et al., Infect Immun. 65(7):2821-8, 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., Vaccine., 16(20):2069-76, 1998); LT7K (Arg to Lys) (Komase et al., Vaccine,16(2-3): 248-54, 1998, Douce et al., Proc Natl Acad Sci U S A. 92(5):1644-8, 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., Vaccine, 16(1):33-7, 1998, Douce et al., 1997, Di Tommaso et al., Infect Immun. 64(3):974-9, 1996); and LTR72 (Ala to Arg) (Giuliani et al., J Exp Med. 187(7):1123-32,1998), Pertussis toxin, PT, including PT-9K/129G (Roberts et al., Infect Immun. 63(6):2100-8, 1995, Cropley et al., Vaccine, 13(17):1643-8, 1995); Toxin derivatives (see below) (Holmgren et al., Vaccine. 11(12):1179-84, 1993, Verweij et al., 1998, Rappuoli et al., Int Arch Allergy Immunol. 108(4):327-33 1995, Freytag and Clements, 1999); Lipid A derivatives (e.g., monophosphoryl lipid A, MPL) (Sasaki et al., Infect Immun. 66(2):823-6, 1998; Muramyl Dipeptide (MDP) derivatives (Fukushima et al., Vaccine. 14(6):485-91, 1996); Bacterial outer membrane proteins (e.g., outer surface protein A (OspA) lipoprotein of Borrelia burgdorferi, outer membrane protein of Neisseria meningitidis) (Marinaro et al., Infect Immun. 67(3):1287-91, 1999, Van de Verg et al., Infect Immun., 64(12):5263-8,1996); Oil-in-water emulsions (e.g., MF59) (Verschoor et al., J. Virol. 73(4):3292-300 1999; O'Hagan, J Pharm Pharmacol.50(1):1-10, 1998); Aluminum salts (Isaka et al., Vaccine. 16(17):1620-6, 1998); and Saponins (e.g., QS21) Aquila Biopharmaceuticals, Inc., Worcester, Mass.) (Sasaki et al., J Virol. 72(6):4931-9, 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Acylated peptides can also be administered to a subject together with a cytokine. Examples of cytokines include, but are not limited to IL-1, IL-2, IL-4, IL-5, IL-6, IL-7, IL-10, IL-12, IL-15, IL-18 granulocyte-macrophage colony stimulating factor (GM-CSF), granulocyte colony stimulating factor (GCSF), interferon-γ (γ-IFN), IFN-a, tumor necrosis factor (TNF), TGF-β, FLT-3 ligand, and CD40 ligand. Cytokines play a role in directing the T cell response. Helper (CD4⁺) T cells orchestrate the immune response of mammals through production of soluble factors that act on other immune system cells, including other T cells. Most mature CD4⁺ T helper cells express one of two cytokine profiles: Th1 or Th2. In some embodiments it is preferred that the cytokine be a Th1 cytokine.

The acylated peptides can also be used as adjuvants given their ability to modulate an immune response. Accordingly, they may be administered together with another antigen (e.g., a CD1 restricted antigen or an MHC restricted antigen) or they may be administered to a subject that is at risk of being exposed to an antigen passively or actively. Subjects that may be passively exposed to an antigen can be one that is in an environment or profession in which exposure to an antigen likely. Examples include being in a country in which particular infectious agents are pandemic, or working in an environment in which infectious agents are common (e.g., a doctor's office or hospital). Active exposure means deliberate exposure to an antigen, such as occurs with a vaccination. Accordingly, the acylated peptides may be used in conjunction with vaccine compositions in order to enhance an immune response to the antigen provided in the vaccine. The vaccines as such can be formulated using a purified antigen or can be formulated using a CD1c-bound antigen. Because CD1-restricted antigens are presented to T cells as a complex of antigen and CD1, the use of an antigen:CD1 complex or an antigen:CD1⁺ cell complex can, in some cases, provide superior immunization properties. A skilled artisan can employ routine formulation procedures in order to formulate an isolated CD1-presented antigen for use as a vaccine. See Remington's Pharmaceutical Sciences, 18th Ed., Gennaro, A. R., ed., Mack, Easton, (1990); The Pharmacologist Basis of Therapeutics, 7th Ed., Gilman, A. G., et al., eds., MacMillan, New York, (1985).

The antigens of the invention can also be used in combination with dendritic cell based vaccines. For example, the antigen can be loaded onto dendritic cells (e.g., autologous dendritic cells), and these cells can then be introduced into a subject. The dendritic cells can also be treated in order to induce CD1c expression.

CD1⁺ cells (e.g. CD1⁺ macrophages) used in the various aspects of the invention can naturally express the CD1 molecule or can be manipulated to do so. For instance, cells can be transfected with an expression vector encoding the CD1 molecule of interest. As used herein, “genetically engineered” refers to any human manipulation intended to introduce genetic change. In this instance, cells can be genetically engineered to express a CD1 molecule. In addition, a cell can also be induced to express CD1 by contacting the cell with one or more cytokines. One skilled in the art can readily vary the contacting time, cytokine type and concentration, and contacting conditions to induce CD1, or in particular, CD1c expression. As used herein, “expressing” refers to the process of producing a gene product by transcription of a DNA molecule to generate a corresponding mRNA molecule that is translated into a polypeptide.

The invention provides methods for modulating immune responses in subjects in need thereof. A “subject” shall mean a human or vertebrate animal including but not limited to a dog, cat, horse, cow, pig, sheep, goat, chicken, non-human primate (e.g., monkey), fish (aquaculture species, e.g., salmon), rabbit, rat, and mouse. A subject in need of immunomodulation may be a subject having or at risk of developing a condition that can be therapeutically benefited by an immune response. Examples of conditions include infections such as bacterial, viral, fungal, and parasitic infections, cancers, allergies, asthma, and the like. A subject having one of these conditions can be readily identified by a medical practitioner as these conditions are known and the symptoms associated with each are also known. A subject at risk of developing one of these conditions is similarly readily identified. Examples include subjects that have been exposed or are likely to be exposed to an infectious organism such as a bacterium, virus, fungus, or parasite. Further examples include subjects that have been exposed or are likely to be exposed to a carcinogen, in the case of cancer. Carcinogens are agents with suspected cancer causing activity.

Acylated peptides can therefore be used to treat subjects having or at risk of developing a condition that could benefit from an immune response. As used herein, the term treat includes prevention of a condition by administering an acylated peptide prophylactically.

Vaccine-induced acquired protective immunity, as used herein, refers to an immunity which occurs as a result of deliberate exposure to an antigen (the compounds of the invention) in a form and dose sufficient to stimulate an immune response to the antigen and, thereby, render the subject immune to subsequent challenge with the antigen. The invention, therefore, provides methods and compositions for enhancing vaccine induced immunity by administering a vaccine comprising an acylated peptide. Methods for enhancing vaccine-induced protective immunity are useful for the treatment or prevention of a variety of diseases including but not limited to infectious disease (i.e., infections).

As used herein, a “subject in need of treatment” includes a subject having an infection, as well as a subject at risk of developing an infection. Additionally or alternatively, a “subject in need of treatment” embraces a subject having an autoimmune disease, as well as a subject at risk of developing an autoimmune disease.

A subject having an infection or an autoimmune disease is a subject with at least one identifiable sign, symptom, or laboratory finding sufficient to make a diagnosis of an infectious disorder or of an autoimmune disease in accordance with clinical standards known in the art for identifying such disorders. Examples of such clinical standards can be found in Harrison's Principles of Internal Medicine, 14th Ed., Fauci A S et al., eds., McGraw-Hill, New York, 1998. In some instances, a diagnosis of an infection will include identification of an infectious organism or agent by culture of the infectious organism or agent from a body fluid or tissue obtained from the subject. Examples of infectious organisms and infectious agents, including but not limited to bacteria, viruses, protozoa, and fungi, are given below.

Examples of infectious bacteria include but are not limited to: Acinetobacter spp., Actinomyces israelli, Bacillus anthracis, Bacteroides spp., Bordetella pertussis, Borrelia burgdorferi, Brucella melitensis, pathogenic Campylobacter spp., Clostridium difficile, Clostridium perfringens, Clostridium tetani, Corynebacterium diphtheriae, other Corynebacterium spp., Enterobacter aerogenes, Enterococcus spp., Erysipelothrix rhusiopathiae, Escherichia coli, Francisella tularensis, Fusobacterium nucleatum, Haemophilus influenzae, Helicobacter pylori, Klebsiella pneumoniae, Legionella pneumophilia, Leptospira spp., Listeria monocytogenes, Mycobacteria spp. (e.g. M. tuberculosis, M. avium, M. gordonae, M. intracellulare, and M. kansasii), Neisseria gonorrhoeae, Neisseria meningitidis, Nocardia asteroides, Nocardia brasiliensis, Pasturella multocida, Peptostreptococcus spp., Proteus spp., Pseudomonas aeruginosa, other Pseudomonas spp., Rickettsia, Salmonella spp., Serratia spp., Shigella spp., Staphylococcus aureus, Streptobacillus moniliformis, Streptococcus (anaerobic spp.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidum, Treponema pertenue, Vibrio cholerae, other Vibrio spp., and Yersinia spp.

Examples of infectious viruses include but are not limited to: Adenoviridae (most adenoviruses); Arena viridae (hemorrhagic fever viruses); Birnaviridae; Hepadnaviridae (Hepatitis B virus); Bungaviridae (e.g., Hantaan viruses, bunga viruses, phleboviruses and Nairo viruses); Calciviridae (e.g., strains that cause gastroenteritis); Coronaviridae (e.g., coronaviruses); Filoviridae (e.g., ebola viruses); Flaviridae (e.g., dengue viruses, encephalitis viruses, yellow fever viruses); Herpesviridae (herpes simplex virus (HSV) 1 and 2, varicella zoster virus, cytomegalovirus (CMV), herpes virus; Iridoviridae (e.g., African swine fever virus); Orthomyxoviridae (e.g., influenza viruses); Paramyxoviridae (e.g., parainfluenza viruses, mumps virus, measles virus, respiratory syncytial virus); Parvovirida (parvoviruses); Papovaviridae (papilloma viruses, polyoma viruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 or HIV-2, or HTLV); Rhabdoviridae (e.g., vesicular stomatitis viruses, rabies viruses); Togaviridae (e.g., equine encephalitis viruses, rubella viruses); and unclassified viruses (e.g., the etiological agents of spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious fungi include but are not limited to: Aspergillus spp., Blastomyces dermatitidis, Candida albicans, other Candida spp., Coccidioides immitis, Cryptococcus neoformans, Histoplasma capsulatum, and Rhizopus spp.

Other infectious organisms include but are not limited to: Plasmodium spp. (e.g., Plasmodium falciparum, Plasmodium knowlesi, Plasmodium malariae, Plasmodium ovale, and Plasmodium vivax), Babesia divergens, Babesia microti, Chlamydia trachomatis, Giardia spp., Leishmania braziliensis, Leishmania donovani, Leishmania major, Leishmania tropica, Toxoplasma gondii, Trichinella spiralis, and Trypanosoma cruzi.

A subject at risk of developing an infection is a subject with an identifiable risk factor for developing an infection. For example, a subject at risk of developing an infection can include an individual with a known or suspected exposure to another individual with an infection (e.g., medical or military personnel). Alternatively, a subject at risk of developing an infection can include an individual with a known or suspected exposure to an agent or vector associated with an infection. Yet other examples of a subject at risk of developing an infection include a subject that is immunocompromised; a subject about to undergo surgery; and a subject that has recently undergone surgery.

A subject that is immunocompromised is a subject with reduced capacity to mount an effective immune response to an infectious agent. Such subjects may have, for example, an immune system that is immature or that is suppressed in association with exposure to certain pharmacological agents, suppressed in association with exposure to irradiation, suppressed in association with a chromosomal defect, suppressed in association with a hereditary or inborn metabolic defect or enzyme deficiency, suppressed in association with an antibody deficiency, suppressed in association with a defect in the ability of T cells to process and/or present antigen, suppressed in association with a nutritional deficiency, suppressed in association with an infection that directly affects cells of the immune system (e.g., HIV), suppressed in association with a neoplasm. These and other examples of conditions that cause a subject to be immunocompromised can be found in Harrison's Principles of Internal Medicine, 14th Ed., Fauci A S et al., eds., McGraw-Hill, New York, 1998.

Thus, in one aspect the invention is useful whenever it is desirable to treat or prevent infection in a subject. This includes prophylactic treatment to prevent such infections in planned surgical procedures, as well as in emergency surgical situations, especially those involving intraabdominal surgeries. Intraabdominal surgeries include, for example: right hemicolectomy; left hemicolectomy; sigimoid colectomy; subtotal colectomy; total colectomy; cholecystectomy; gastrectomy; nephrectomy; vascular repair, including resection of abdominal aortic aneurysm; abscess drainage. Emergency surgeries include, in addition to any of the above, those to correct the following conditions: perforated ulcer (duodenal or gastric); perforated diverticulitis; obstructive diverticulitis; acute appendicitis; perforated appendicitis; blunt abdominal trauma; penetrating abdominal trauma; ruptured abdominal aorta, second operation to drain abscess; etc. The invention also is useful with non-intraabdominal surgeries such as orthopedic surgeries, pelvic and gynecologic surgeries, urologic surgeries, cardiothoracic surgeries, neurosurgeries, plastic and reconstructive surgeries, vascular surgeries, head and neck surgeries, and surgeries to correct wound infections. These listed surgeries are provided only by way of example and are not intended to be limiting.

A subject about to undergo surgery can be a subject scheduled to undergo an elective or non-emergency surgical procedure. Alternatively, a subject about to undergo surgery can be a subject about to have surgery on an emergency basis. Typically, a subject about to undergo surgery includes a subject that is to have a surgical procedure within the next 24 to 48 hours. A subject about to undergo surgery can include a subject that is to have a surgical procedure within the next 2 to 14 days.

A subject that has recently undergone surgery typically includes a subject that already had a surgical procedure in the previous 24 to 48 hours.

The antigens may be administered alone (e.g., in saline or buffer) or using any delivery vehicle known in the art. For instance, the following delivery vehicles have been described: cochleates (Gould-Fogerite et al., AIDS Res Hum Retroviruses. 10 Suppl 2:S99-103, 1994); emulsomes (Vancott et al., J Immunol. 15;160(4):2000-12, 1998); ISCOMs (Mowat et al., Immunology. 80(4):527-34, 1993; Carlsson et al., Vaccine. 9(8):577-80, 1991); liposomes (Childers et al., Infect Immun. 67(2):618-23, 1999; Michalek et al., Curr Top Microbiol Immunol. 146:51-8, 1989); live bacterial vectors (e.g., Salmonella, Escherichia coli, Bacillus Calmette-Guerin, Shigella, Lactobacillus) (Hone et al., J Biotechnol. 44(1-3):203-7, 1996; Pouwels et al., Int J Food Microbiol. 41(2):155-67, 1998; Chatfield et al., FEMS Immunol Med Microbiol. 7(1):1-7, 1993); live viral vectors (e.g., Vaccinia, adenovirus, Herpes Simplex) (Gallichan et al., J Infect Dis. 168(3):622-9, 1993; 1995; Flexner et al., Virology. 166(2):339-49, 1988, Morrow et al., Curr Top Microbiol Immunol. 1999;236:255-73, 1999); microspheres (Gupta et al., Dev Biol Stand. 92:63-78, 1998; Jones et al., J Biotechnol. 44(1-3):29-36, 1996); nucleic acid vaccines (Fynan et al., Proc Natl Acad Sci U S A. 90(24):11478-82, 1993; Kuklin et al., J Virol. 71(4):3138-45 1997; Sasaki et al., J Virol. 72(6):4931-9, 1998; Okada et al., J Immunol. 159(7):3638-47, 1997; Ishii et al., Microbiol Inuunol. 41(5):421-5, 1997); polymers (e.g., carboxymethylcellulose, chitosan) (Hamajima et al., Clin Immunol Immunopathol. 88(2):205-10, 1998; Jabbal-Gill et al., 1 Vaccine.16(20):2039-46, 1998); polymer rings (Wyatt et al., J Control Release. 50(1-3):93-102, 1998); Proteosomes (Lowell et al., J Infect Dis. 175(2):292-301, 1997); sodium fluoride; transgenic plants (Tacket et al., Nat Med. 4(5):607-9,1998; Mason et al., Vaccine.16(13):1336-43, 1998); virosomes (Gluck et al., 1: J Clin Invest. 90(6):2491-5, 1992; Cryz et al., Vaccine. 15(15):1675-9, 1997); virus-like particles (Jiang et al., Vaccine. 17(19):2461-71, 1999). Those skilled in the art will recognize that other delivery vehicles that are known in the art may also be used.

Combined with the teachings provided herein, by choosing among the various antigens and their intended use, and weighing factors such as potency, relative bioavailability, patient body weight, severity of adverse side-effects and preferred mode of administration, an effective therapeutic treatment regimen can be planned which does not cause substantial toxicity and yet is entirely effective to treat the particular subject as described above. The effective amount for any particular application can vary depending on such factors as the disease or condition being treated, the particular agent being administered, whether a secondary antigen is also administered and the nature of such antigen (e.g., when the agents are used as adjuvants), the size of the subject, or the severity of the disease or condition. One of ordinary skill in the art can empirically determine the effective amount of a particular acylated peptide and/or other therapeutic agent without necessitating undue experimentation.

For adult human subjects, doses of the acylated peptides typically range from about 50 μg/dose to 20 mg/dose, more typically from about 80 μg/dose to 8 mg/dose, and most typically from about 800 μg/dose to 4 mg/dose. Stated in terms of subject body weight, typical dosages range from about 0.5 to 500 μg/kg/dose, more typically from about 1 to 100 μg/kg/dose, and most typically from about 10 to 50 μg/kg/dose. Doses will depend on factors including the route of administration, e.g., oral administration may require a substantially larger dose than subcutaneous administration.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients. Compositions that comprise a pharmaceutically acceptable carrier are generally referred to herein as pharmaceutical compositions.

The peptides can be together with other therapeutic agents known in the art to be useful in treating particular conditions. When administered together with another therapeutic agent, an acylated peptide can be administered before, with or after administration of the other therapeutic agent.

For example, acylated peptides can be administered in combination with anti-bacterial agents, anti-viral agents, anti-fungal agents, and anti-parasitic agents.

Anti-bacterial antibiotic drugs are well known and include, for example: amdinocillin, amikacin, aminoglycosides, amoxicillin, ampicillin, avlocillin, azithromycin, bacampicillin, carbenicillin, cefaclor, cefadoxil, cefamandole, cefazolin, cefinenoxine, cefonicid, cefoperazone, cefotaxime, cefotetan, cefoxitin, ceftazidme, ceftizoxime, ceftriaxone, cefuroxime axetil, cephalexin, cephradine, chloramphenicol, clavulanate, clindamycin, cloxacillin, cyclacillin, dicloxacillin, epicillin, erythromycin, flucloxacillin, gentamicin, hetacillin, imipenem, lincomycin, methicillin, metronidazole, mezlocillin, moxalactam, nafcillin, neomycin, oxacillin, penicillin G, penicillin V, piperacillin, pivampicillin, quinolones, rifampin, sulbactam, tetracyclines, ticarcillin, timentin, tobramycin, trimethoprim-sulfamethoxazole, and vancomycin. (See Goodman and Gilman's The Pharmacological Basis of Therapeutics, 9th Ed., 1996, McGraw Hill, Inc.)

Anti-virals include, for instance, but are not limited to acemannan; acyclovir; acyclovir sodium; adefovir; alovudine; alvircept sudotox; amantadine hydrochloride; aranotin; arildone; atevirdine mesylate; avridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; interferon alpha (IFN-α); kethoxal; lamivudine; lobucavir; memotine hydrochloride; methisazone; nevirapine; penciclovir; pirodavir; ribavirin; rimantadine hydrochloride; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; and zinviroxime.

Anti-fungals include, for instance, but are not limited to acrisorcin; ambruticin; amphotericin B; azaconazole; azaserine; basifungin; bifonazole; biphenamine hydrochloride; bispyrithione magsulfex; butoconazole nitrate; calcium undecylenate; candicidin; carbol-fuchsin; chlordantoin; ciclopirox; ciclopirox olamine; cilofungin; cisconazole; clotrimazole; cuprimyxin; denofungin; dipyrithione; doconazole; econazole; econazole nitrate; enilconazole; ethonam nitrate; fenticonazole nitrate; filipin; fluconazole; flucytosine; fungimycin; griseofulvin; hamycin; isoconazole; itraconazole; kalafungin; ketoconazole; lomofungin; lydimycin; mepartricin; miconazole; miconazole nitrate; monensin; monensin sodium; naftifine hydrochloride; neomycin undecylenate; nifuratel; nifurmerone; nitralamine hydrochloride; nystatin; octanoic acid; orconazole nitrate; oxiconazole nitrate; oxifungin hydrochloride; parconazole hydrochloride; partricin; potassium iodide; proclonol; pyrithione zinc; pyrrolnitrin; rutamycin; sanguinarium chloride; saperconazole; scopafungin; selenium sulfide; sinefungin; sulconazole nitrate; terbinafine; terconazole; thiram; ticlatone; tioconazole; tolciclate; tolindate; tolnaftate; triacetin; triafungin; undecylenic acid; viridofulvin; zinc undecylenate; and zinoconazole hydrochloride.

The peptide antigens can be administered with anti-microbial antibodies such as but not limited to cytomegalovirus immune globulin, GAMIMUNE® N (Bayer), hepatitis B immune globulin, rabies immune globulin, and Varicella-Zoster immune globulin.

The peptide antigens can also be administered with GM-CSF and IL-4 or crude mycobacterial wall preparations such as Freund's adjuvants.

### Pharmaceutical Compositions

The acylated peptides can be administered to a subject by any mode that delivers them to the desired site, e.g., mucosal, systemic. “Administering” the pharmaceutical composition of the present invention may be accomplished by any means known to the skilled artisan. Preferred routes of administration include but are not limited to oral, parenteral, intralesional, topical, transdermal, intramuscular, intranasal, intratracheal, inhalational, ocular, vaginal, and rectal.

For oral administration, the agents can be formulated readily by combining with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer Science 249:1527 (1990), which is incorporated herein by reference.

The acylated peptide agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulfuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2 percent w/v); citric acid and a salt (1-3 percent w/v); boric acid and a salt (0.5-2.5 percent w/v); and phosphoric acid and a salt (0.8-2 percent w/v). Suitable preservatives include benzalkonium chloride (0.003-0.03 percent w/v); chlorobutanol (0.3-0.9 percent w/v); parabens (0.01-0.25 percent w/v) and thimerosal (0.004-0.02 percent w/v).

The pharmaceutical compositions of the invention contain a pharmaceutically-acceptable carrier. The term “pharmaceutically-acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

A variety of administration routes are available. The particular mode selected will depend, of course, upon the particular adjuvants or antigen selected (depending upon the method employed), the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer-based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### Screening Methods and Diagnostics

The identification and purification of a CD1c-presented antigen facilitates the identification of further antigens that bind to CD1c. One method provided by the invention is a screening method for other CD1c presented antigens based on the ability of a compound to compete with, for example, a Nex antigen described herein, for binding to CD1c. Alternatively, variants a Nex antigen can be synthesized and tested for their ability to compete with binding of, for example, Nex2. Compounds so identified may be either agonists or antagonists, depending upon their effect on T cell stimulation following CD1c binding. If the compound is able to compete with Nex2 for binding to CD1c and also activate CD1c restricted T cells, then the compound would be an agonist. If on the other hand it is able to compete with Nex2 but not activate CD1c restricted T cells, then it would be an antagonist.

Accordingly, the present invention further provides inhibitors of CD1c-restricted antigen presentation to T cells, i.e., CD1c blocking agents. A “CD1 blocking agent” is a composition or compound which is capable of blocking the interaction of a CD1-presented antigen with CD1, or of blocking the interaction between CD1: antigen complexes and their cognate T cell receptors. Blocking agents include (1) agents which bind to CD1, (2) agents which bind to a CD1-presented antigen, (3) agents which bind to a CD1:antigen complex, (4) agents which bind to a T cell receptor that recognizes a CD1:antigen complex and (5) agents which prevent the processing of a CD1-presented antigen. In preferred embodiments, the CD1 blocking agents of the invention are CD1c blocking agents and even more preferably, they function by competing with CD1c binding to the CD1c presented antigens of the invention. That is, the agonists and antagonists identified according to the invention are preferably identified by their ability to either substitute for or inhibit the effects of the CD1c presented antigens of the invention.

CD1c antigen presentation can be inhibited by using a CD1c blocking agent to block the ability of a CD1c-restricted antigen to bind to CD1c. As used herein, a CD1c blocking agent is said to “inhibit CD1c-restricted antigen presentation” when the CD1c blocking agent decreases (1) the binding of a CD1c-presented antigen to a CD1c molecule or (2) the binding of a CD1c:CD1c-presented antigen complex to its cognate T cell receptors. Some CD1c blocking agents are able to block such binding to undetectable levels while other CD1c blocking agents only slightly decrease such binding. CD1c blocking agents include (1) agents which bind to CD1c, (2) agents which bind to the CD1c-presented antigen, (3) agents which bind to the CD1c:antigen complex, and (4) agents which bind to the T cell receptors that recognize the CD1c:antigen complex. Respective examples of blocking agents include, but are not limited to, (1) polyclonal or monoclonal antibodies which bind to and block the portion of a CD1c molecule that binds a CD1c-presented antigen, (2) polyclonal or monoclonal antibodies which bind to and block the portion of a CD1c-presented antigen that binds CD1c, (3) synthetic oligopeptides that are derived from the CD1c:antigen-binding portion of a T cell receptor and which bind to and block the portion of the CD1c:antigen complex bound by intact T cell receptors, and (4) synthetic compounds comprising a CD1c-presented antigen chemically linked to a purified CD1c molecule or a synthetic derivative thereof.

In an alternative method for inhibiting antigen presentation of CD1c-restricted antigens, a CD1c blocking agent can be employed which blocks the interaction of the antigen:CD1c complex with the TCR molecules on the T cell. By inhibiting the presentation step, the activation of specific subsets of T cells can be inhibited. DNA molecules encoding TCR polypeptides displayed by T cells that recognize the CD1c-presented antigens of the invention are isolated according to methods known in the art. Oskenberg, I. R., et al., Proc. Natl. Acad. Sci. USA 86:988-992, 1989; Oksenberg, J. R., et al., Nature 345:344-346,1990 and erratum, Nature 353:94,1991; Uematsu, Y., et al., Proc. Natl. Acad. Sci. USA 88:534-538, 1991; Panzara, M. A., et al., Biotechniques 12:728-735,1992; Uematsu, Y., Immunogenet. 34:174-178,1991. The DNA sequence is converted into a polypeptide sequence, and the portion of the polypeptide sequence that corresponds to the antigen-binding variable region of a TCR polypeptide is used to design synthetic oligopeptides that bind CD1c:antigen complexes on APCs, thereby inhibiting antigen presentation. Oligopeptides are chemically synthesized according to standard methods (Stewart and Young, Solid Phase Peptide Synthesis, Pierce Chemical Co., Rockland, Ill., 1985) and purified from reaction mixtures by reversed phase high pressure liquid chromatography (HPLC). Additionally or alternatively, methods for generating anti-TCR antibodies and anti-TCR binding peptides are well known in the art with regard to MHC presentation and can readily be adapted to the herein disclosed CD1c presentation system. Strominger, J. L., Cell 57:895-898,1989; Davis, M. M., and Bjorkman, P. J., Nature 334:395-404, 1989.

A skilled artisan can readily employ known methods of antibody generation, as well as rational blocking agent design in order to obtain the blocking agents of the present invention. Harlow, E., and Lane, D., Antibodies: A Laboratory Manual, Cold Spring Harbor Press, Cold Spring Harbor, 1988; Synthetic Peptides: Answers Guide, Freeman, W. H., New York, 1991; Kasprzak, A. A., Biochemistry 28:9230-9238, 1989. Additionally or alternatively, libraries of molecularly diverse molecules can be screened for individual member molecules which are CD1c blocking agents. Effective CD1c blocking agents are identified by their ability to inhibit CD1c-mediated T cell proliferative and/or cytolytic responses using the materials and methods described herein.

CD1-presented acylated peptides can be employed in diagnostic assays to evaluate an individual's immune response to an antigen. For example the acylated peptides described herein can be used in assays of the potential or capacity of a subject to mount a cell-mediated immune (CMI) response. The assay can be based on the measurement of proliferation or immune effector molecule production by cells of the immune system in response to stimulation with an acylated peptide antigen. The immune effectors may be detected using ligands such as antibodies specific for the effectors or by measuring the level of expression of genes encoding the effectors. Means for evaluating cell proliferation are well-known in the art. These assays can be used as a means for the diagnosis of infectious diseases, pathological conditions, level of immunocompetence and a marker of T cell responsiveness to endogenous or exogenous antigens.

Methods for evaluating immune responsiveness can include the following steps. Briefly, a sample is collected from the subject to be evaluated. The sample includes cells of the immune system (e.g., T cells) which are capable of proliferating or producing immune effector molecules (e.g., cytokines) following stimulation by an antigen. The sample is incubated with an antigen (e.g., an acylated peptide antigen) and the presence of or elevation in proliferation or the level of an immune effector molecule is determined. The presence or level of proliferation or immune effector molecule is indicative of the capacity of the subject to mount a cell-mediated immune response, and/or may indicate past exposure to the antigen of interest. Various assays for measuring CMI responses are known in the art. See, e.g., U.S. Pat. Pub. No. 20050014205.

### EXAMPLES

### Introduction

In addition to lipids and glycolipids, CD1 proteins present acylated peptide antigens to T cells. It had previously been shown that a CD1a-presented lipopeptide, didehydroxymycobactin (DDM), is produced by non-ribosomal peptide synthases and contains an unusual acyl-lysine unit (Moody et al., Science, 303(5657):527-3 1, 2004). The experiments described below report the identification of a novel synthetic lipopeptide T cell antigen, Nex. This antigen is presented by CD1c and recognized in a TCR-mediated manner by human T cell lines and clones. In contrast to DDM, Nex contains an N-terminally acylated glycine. The lipid linkage found in Nex is chemically identical to that of many types of self and viral lipoproteins that are synthesized by ribosomes.

Dideoxymycobactin (DDM) is involved in scavenging iron from cells infected with mycobacteria. The mechanism of presentation involves anchoring antigens in the hydrophobic binding groove, resulting in exposure of the peptide moiety for TCR contact. This finding showed that CD1, like MHC class I and class II molecules, allows T cells to discriminate among peptide sequences. DDM and the related molecule mycobactin are synthesized by non-ribosomal peptide synthases (NRPS). NRPS pathways use conventional enzymes, in this case mycobactin synthases (MbtA and related proteins), to couple amino and organic acids together. The use of NRPS limits the degree of molecular diversity of a certain class of molecules. Unlike ribosomes, which use transfer RNA-amino acids as substrates to produce polypeptides of varying sequence, NRPS show substrate specificity for individual amino acids, so that they produce peptides of invariant or nearly invariant sequence. Another structural difference with ribosomally synthesized peptides is that NRPS-produced peptides are not linked by classical peptide bonds.

Nex is a novel synthetic antigen that resembles a ribosomal lipopeptide in that it has an N-terminal acylation and classical peptide bonds. Nex is the first example of a lipopeptide presented by CD1c.

### RESULTS

First, T cell lines were derived by stimulation with monocyte-derived dendritic cells (DCs) and a mixture of lipopeptides that were synthesized using standard solid phase peptide synthesis techniques followed by N-terminal fatty acylation. Lipopeptides were synthesized by N-acylation of a peptidic backbone (GGKWSK; SEQ ID NO: 1) with a mixture of fatty acids (C14:0, C16:0, C18:0, C18:1, C20:0) (Anaspec corporation).

To isolate CD1-restricted T cells, polyclonal human lymphocytes were stimulated three times with synthetic antigen mixtures at a concentration of 1 μg/ml, and autologous DC at 2-week intervals, followed by stimulations with heterologous DC and antigen. For T cell proliferation assays, 1.25×10⁵ T cells and 0.25×10⁵ DC were plated per well in round bottom 96-well plates. T cell medium was made by supplementing 500 ml of RPMI medium with 50 ml fetal calf serum (Hyclone), penicillin (Gibco), streptomycin (Gibco), 20 mM HEPES (Gibco), and 4 ml 1 N NaOH solution. The IL-2 concentration was gradually increased from 0.1 nM to 1 nM during subsequent rounds of stimulation.

T cell clones were derived by limiting dilution, using 0.6×10⁵ EBV transformed B cells (10,000 R) and 1.3×10⁵ heterologous PBMC (3300 R) as feeder cells and 1 82 g/ml PHA (Difco) in medium containing 2 nM IL-2. T cell activation was measured by incubating 5×10⁴ T cells with 3×10⁴ DC or CD1-transfected C1R cells. Proliferation was measured after coculture for 3 days with antigen, followed by a 6 h pulse of 1 μCi of [³H]thymidine before harvesting and counting β emissions.

The IL-2 concentration in T cell culture supernatants was determined by harvesting culture supernatants 24 hours after T cell stimulation, adding IL-2 starved HT2 cells in medium, and measuring proliferation of HT2 cells after a 6 h pulse with 1 μCi of [³H]thymidine before harvesting and counting β emissions.

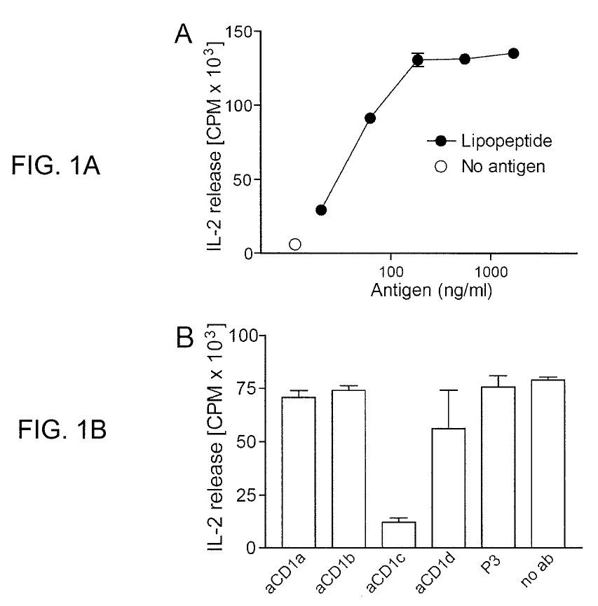

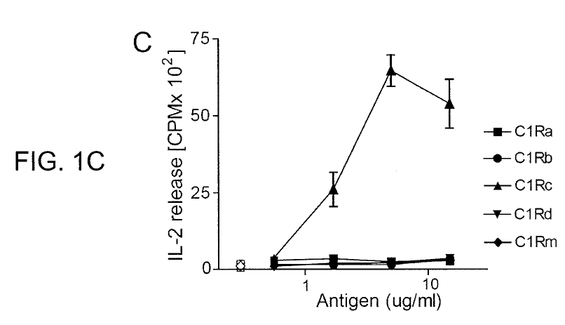

One of the resulting T cell lines, named Next, was specific for synthetic antigen. Antigen-specific IL-2 release by Next T cells is depicted in FIG. 1A. To determine the CD1-dependence of the Next T cell responses, cells were stimulated with DC and antigen in the presence of monoclonal antibodies against CD1a, CD1b, CD1c, CD1d, or isotype matched control (P3) (20 μg/ml). Antibodies were added prior to adding the antigen mixture at 0.5 μg/ml. Next T cell stimulation was dependent upon CD1c, as blocking mAb against CD1c inhibited the response (FIG. 1B). Antibodies against CD1a, CD1b, and CD1d did not inhibit IL-2 release.

Further assays were carried out in which CD1-deficient C1R B lymphoblastoid cells transfected with human CD1 proteins were employed as APC. C1R cells were treated with the antigen mixture prior to adding polyclonal Next T cells and measuring IL-2 release. IL-2 release was observed only when CD1c-transfected C1R cells were used as APC (FIG. 1C). C1R cells transfected with CD1a, CD1b, or CD1d did not stimulate IL-2 release (FIG. 1C). These data show that antigen recognition was absolutely dependent on CD1c expression and could not be carried out by CD1a, CD1b or CD1d.

### Characterization of T Cell Receptor Expression

The T cell receptor (TCR) variable segments expressed by the Nex-reactive cells were analyzed by PCR using a set of primers that covered most of the variable segments. To analyze the variable gene segments, mRNA was isolated from 10⁶ clonal or polyclonal T cells using an Oligotex Direct mRNA kit (Qiagen), followed by first strand cDNA synthesis using SuperScript RT (Invitrogen). PCR-primer sets which amplify most of the TCR Vα and Vβ families were used to determine the Vα and Vβ usage. These primer sets are described on the Immunogenetics website (available on the World Wide Web at imgt.cines.fr). Second strand cDNA synthesis was performed using E. coli DNA ligase (Invitrogen), E. coli DNA pol I (Invitrogen) Rnase H (New England Biolabs) in E. coli ligase buffer (Invitrogen), followed by blunting of the material with T4 polymerase and circularization using T4 ligase. Inverse PCR was performed using the following primers:

(SEQ ID NO: 15)

| | |
|---|---|
| CircularCaFor: | GACCTCATGTCTAGCACAGTTTTG; |

(SEQ ID NO: 16)

| | |
|---|---|
| CircularCaRev: | GCCCTGCTATGCTGTGTGTCT; |

(SEQ ID NO: 17)

| | |
|---|---|
| CircularCbFor: | ACACAGCGACCTCGGGAGGG; |

(SEQ ID NO: 18)

| | |
|---|---|
| CircularCbRev: | GATGGCCATGGTCAAGAGAAAGGA. |

Primers used to amplify full length TCR chains were:

| | |
|---|---|
| FLTRBV12-3: | |
| GCCATGGACTCCTGGACCTTCTGCT; | (SEQ ID NO: 19) |
| and | |
| FLTRAV25: | |
| GGGAGATGCTACTCATCACATCAATGTTG. | (SEQ ID NO: 20) |

A strong, reproducible positive PCR signal was observed for the TRBV12-3 segment of the β chain. A positive signal was not observed for the (α chain in this initial experiment. Because the PCR primer sets did not examine all possible variable segments, inverse PCR was performed on circularized cDNA followed by cloning and sequencing of the PCR product. The initial finding that Nex uses the TRBV12-3 segment was confirmed. The α chain PCR product was identified as TRAV25. This variable segment was not covered by the initial PCR primer sets. The full length TCR α and β chain were cloned from clone 1A3 and sequenced.

### Characterization of Nex Antigens and Comparison to DDM

To identify the antigenic component of the lipopeptide mixture, antigenic compounds were fractionated by high performance liquid chromatography with a split interface to allow for simultaneous preparative collection of samples for T cell assays, electrospray ionization mass spectrometry (Thermo Electron Corporation) and UV detection (254 and 280 rm). A Vydac C18 column was used for fractionation with a gradient elution based on solvent A (80:20 v/v water:acetonitrile with 0.02% trifluoroacetic acid, 0.1% formic acid) and solvent B (50:30:20 methanol:acetonitrile:water with 0.02% triflouroacetic acid and 0.1% formic acid) using a flow rate of 0.7 ml/min and a gradient starting at 50% B and running to 95% B over 20 min and holding at 95% B for final 10 min. More detailed MS experiments were carried out using nanoelectrospray ionization using borosilicate glass pipets pulled to a final orifice of 1-2 μm and an internal stainless steel electrode on the same ion trap mass spectrometer as above.

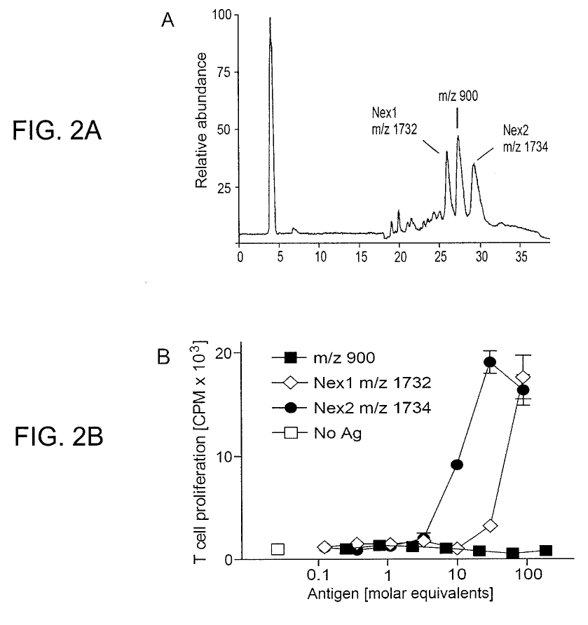

The antigen mixture contained three major compounds with a m/z of 1732 (Nex1), 900, and 1734 (Nex2) (FIG. 2A). The proliferative response of Next T cells was analyzed by stimulating 5×10⁴ Next T cells with 3×10⁴ DC and antigen by coculture for 3 days, followed by a 6 h pulse of 1 μCi of [³H]thymidine, before harvesting and counting β emissions. Antigen quantities were normalized for absorbance at 280 nm. Two of the three major compounds, Nex1 and Nex2, were recognized by the T cell clone 1A3 (FIG. 2B).

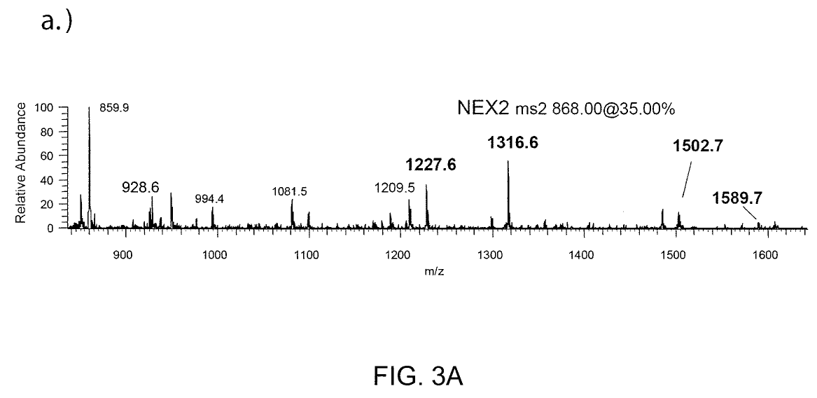

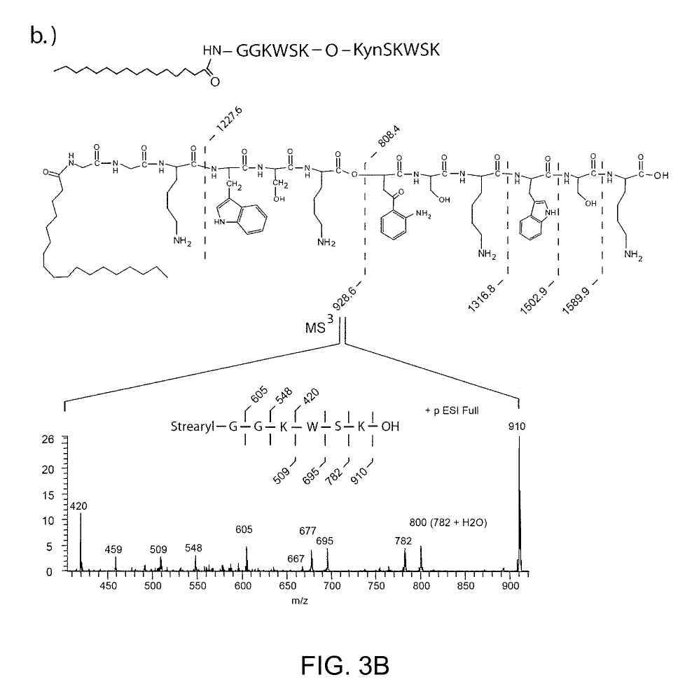

Collisional mass spectrometry (FIG. 3A) led to the proposed structure of Nex2 as shown in FIG. 3B. Nex1 and Nex2 are related structures, differing only by an unsaturation in the fatty acid.

For biological testing, fractions were collected at 15 second intervals with an automatic fraction collector, evaporated to dryness under nitrogen and tested for stimulation of T cells. DDM was purified from M. tuberculosis as described (Moody et al., Science, 303(5657):527-31, 2004).

The Nex antigen is the second peptidic antigen shown to be presented by CD1. The other one, DDM, is presented by CD1a. Although processing of proteins for presentation on MHC Class I and Class II molecules is well characterized, little is known about processing of CD1-presented peptides. The presence of a peptide backbone of amino acids coupled by peptide bonds suggest that Nex can be subject to proteolytic breakdown by non-specific proteases like proteinase K and pronase. DDM has no such cleavage sites for proteases. To examine the protease sensitivity of Nex, Nex and DDM were treated with non-specific proteases and T cell stimulation by each antigen was examined.

Pronase is a mixture of endopeptidases and exopeptidases (carboxypeptidases and aminopeptidases) which digests denatured and native proteins into individual amino acids. Proteinase K is an endopeptidase with a preference for cleavage between an aliphatic, aromatic, or hydrophobic and any other amino acid, however, it will digest any peptidic bond if added in excess and over long incubation periods. Pronase and proteinase K were used to digest Nex and DDM in protease buffer (10 mM CaCl, 10 mM HEPES buffer, 25 mM ammonium bicarbonate) for 4 hours at 40° C., followed by 10 minute inactivation at 85° C.

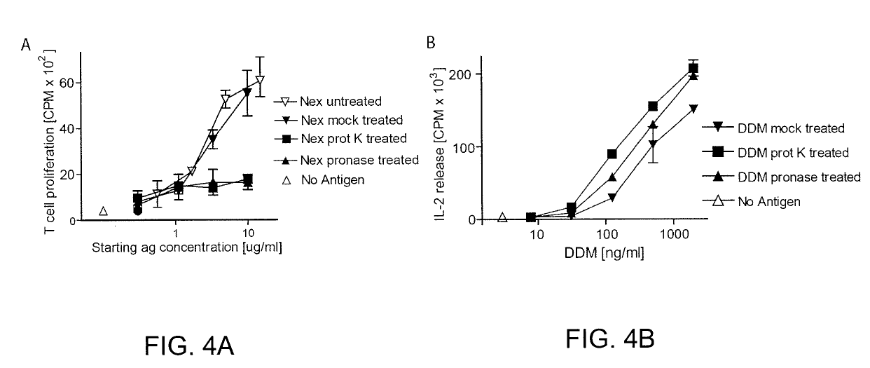

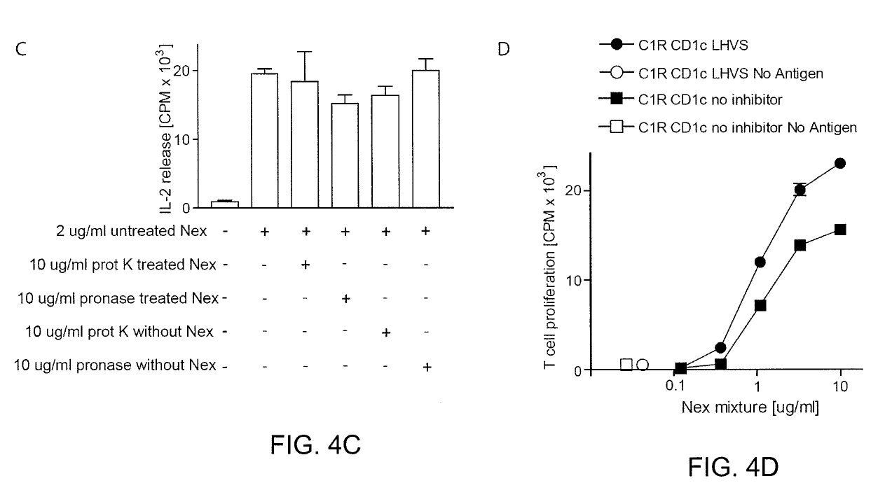

The results show that protease treatment did not diminish the ability of DDM to stimulate T cell proliferation (FIG. 4B). In contrast, the activity of Nex was greatly reduced by protease treatment (FIG. 4A). To test whether treated Nex mixtures were toxic to T cells, T cell stimulation was measured in the presence of a high concentration (10 μg/ml) of treated Nex and a suboptimal concentration of untreated Nex. The results, shown in FIG. 4C, indicate that protease treated Nex is not toxic to T cells. FIG. 4D depicts proliferative responses in the presence and absence of of LHVS, a protease inhibitor.

Nex is an antigen that resembles ribosomally-produced peptides modified by N-myristoyl transferase in that the linkage of the N-terminal acylation and certain six amino acids are chemically identical to naturally occurring lipoproteins. The discovery of Nex provides a new insight into the function of CD1 and indicates a novel class of T cell antigens which include N-terminal acylation. The fatty acids that are part of Nex1 (C18:0) and Nex2 (C18:1) are atypical. Most N-terminal acylations in eukaryotes are N-myristoylations (C14), but C16 and C18 fatty acids also occur.

Mannosyl phosphoisoprenoids (MPI) are a second class of structures presented by CD1c molecules. For the recognition of MPI, the hydrophylic head and the saturation of the proximal cc-isoprene unit are important for T cell recognition. The structure of Nex resembles MPI in the sense that it has a single lipid tail and a hydrophilic portion, as is the case for most known CD1-presented antigens. Based on the mode of recognition of MPI, the cocrystal of DDM lipopeptide and CD1a, and on the other available structures of ligand-bound CD1 molecules, it is proposed that Nex binds with its acylation in the antigen binding groove of CD1c. This mode of binding would leave part of the peptide available for TCR recognition.

Modified tryptophan residues like the one found in Nex are not used during ribosomal protein synthesis. Nex can thus never be expressed in the thymus during negative selection of T cells, which may explain why T cells that recognize Nex have not been deleted. If negative selection plays role in shaping the repertoire of CD1c restricted T cells, as it does for CD1d restricted T cells, the repertoire would include T cells that recognize N-terminally acylated proteins encoded by pathogens and that possess sequences that differ from the sequences of self lipoproteins.

All CD1 presented antigens known so far are not directly encoded by a host or pathogen genome, but are generated by series of enzymatic steps. This had led to the notion that CD1 presented antigens are always conserved structures. The data presented here indicate that this may not be a general rule, and that ribosomally produced peptides, which are subject to mutations, may be presented by CD1.

These studies expand the known reactivity of CD1 to include N-terminally acylated peptides. N-terminally acylated proteins are found in eukaryotes, and are involved in many cellular processes, including intracellular signaling (α subunit of G-proteins), cell cycle control (Src family members). Viral genomes encode lipoproteins, including Nef genes, present in several viruses, that are important for viral budding. Importantly, Nef loses its function and renders HIV non infectious if it is mutated in a way that prevents N-terminal acylation. These findings describe an enormous new class of antigens presented by CD1.

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a compound of formula I: R-GGKWSK (SEQ ID NO: 2);
wherein R is an alkyl or alkene chain.

2. A composition comprising a compound of formula III: R-GGKWSK-O-KynSKWSK (SEQ ID NO: 3); wherein R is an alkyl or alkene chain.

3. The composition of claim 2, wherein R is an alkyl or alkene chain at least 12, 13, 14, 15, 16, 17 or 18 carbons in length.

4. The composition of claim 2, wherein R is one of the following (number of carbons: number ofunsaturated bonds): C20, C20:1, C19, C19:1, C18, C18:1, C17, C17:1, C16, C16:1, C15, C15:1, C14, C14:1, C13, C13:1, C12 and C12:1.

5. A composition comprising a glycine-acylated peptide, wherein the peptide is present in an amount sufficient to modulate proliferation of a T cell.

6. The composition of claim 5, wherein the peptide is N-terminally acylated.

7. The composition of claim 5, wherein the peptide is present in an amount sufficient to modulate an immune response in a subject.

8. The composition of claim 5, wherein the peptide stimulates T cell proliferation.

9. The composition of claim 5, wherein the peptide inhibits T cell proliferation.

10. The composition of claim 5, wherein the peptide is a compound of formula V: R-G-Xₙ; wherein R is an alkyl or alkene, wherein G is glycine and wherein X is any amino acid.

11. The composition of claim 5, wherein the peptide comprises at least 3 amino acid residues.

12. The composition of claim 5, wherein the peptide is 5-10 amino acid residues in length.

13. The composition of claim 5, wherein the peptide is a glycine-acylated peptide which is 6 amino acids in length.

14. The composition of claim 5, wherein the peptide is acylated with an alkyl or alkene chain.

15. The composition of claim 13, wherein alkyl or alkene chain is at least 12, at least 13, at least 14, at least 15, at least 16, at least 17 or at least 18 carbons in length.

16. The composition of claim 13, wherein the alkyl or alkene chain is one of the following (number of carbons: number of unsaturated bonds): C20, C20:1, C19, C19:1, C18, C18:1, C17, C17:1, C16, C16:1, C15, C15:1, C14, C14:1, C13, C13:1, C12 and C12:1.

17. The composition of claim 5, wherein the peptide is a microbial peptide or fragment thereof.

18. The composition of claim 5, wherein the peptide is a mammalian peptide or fragment thereof.

19. The composition of claim 5, further comprising a second compound.

20. The composition of claim 19, wherein the second compound comprises a second peptide or non-peptide antigen.

21. The composition of claim 19, wherein the second compound comprises an immunomodulatory agent selected from a cytokine, an adjuvant, or an immunosuppressive drug.

22. An immunogenic composition comprising a glycine-acylated peptide, wherein the peptide is present in an amount sufficient to modulate proliferation of a T cell.

23. A method for modulating activity of a T cell, the method comprising:
contacting the T cell with a composition comprising an antigen-presenting cell is (APC) and a glycine-acylated peptide, thereby modulating the activity of the T cell.

24. The method of claim 23, wherein the APC expresses CD1 molecules.

25. The method of claim 23, wherein the peptide is N-terminally acylated.

26. The method of claim 23, wherein the peptide is a compound of formula V: R-G-Xₙ; wherein R is an alkyl or alkene, wherein G is glycine, and wherein X is any amino acid.

27. The method of claim 23, wherein the acylated peptide is a compound of formula I, II, III, or IV.

28. The method of claim 23, wherein T cell activity is modulated in a subject in vivo or ex vivo or in vitro.

29. The method of claim 23, wherein the subject is at risk for, being screened for or diagnosed with an infection, an autoimmune disorder, an allergic disorder, or a neoplastic disorder.

30. The method of claim 29, wherein the subject is at risk for, being screened for or diagnosed with an infection.

31. A method for modulating an immune response in a subject, the method comprising:
identifying a subject in need of modulation of an immune response,
administering to the subject a composition comprising a glycine-acylated peptide in an amount effective to modulate an immune response.

32. An isolated CD1-reactive T cell, wherein the CD1-reactive T cell is specific for an acylated peptide.

33. The T cell of claim 32, wherein the T cell is CD1c-reactive.

34. The T cell of claim 32, wherein the T cell comprises an αβ T cell receptor.

35. A method for identifying a T cell antigen, the method comprising:
providing a sample comprising an antigen-presenting cell (APC), wherein the APC expresses CD1 molecules;
contacting the sample with a composition comprising a glycine-acylated peptide under conditions in which acylated peptides bind to CD1 molecules;
contacting the sample with a CD1-restricted T cell; and
determining activity of the T cell in the presence of the sample, wherein a change in activity of the T cell in the presence of the sample, relative to a control, indicates that the peptide is a T cell antigen.

36. The method of claim 35, wherein the T cell is a CD1c-restricted T cell.

37. The method of claim 35, further comprising purifying the peptide from the composition.

38. A method for evaluating an immune response in a subject, the method comprising:
providing a sample from the subject, wherein the sample comprises T cells;
contacting the sample with a composition comprising an antigen-presenting cell (APC) and a glycine-acylated peptide;
evaluating activity of the T cells, relative to a control, thereby evaluating an immune response in the subject.

39. The method of claim 38, wherein the immune response of the subject to a microbe is evaluated.

40. The method of claim 38, wherein the immune response of the subject to an allergen is evaluated.

41. The method of claim 38, wherein the immune response of the subject to a self antigen is evaluated.
